(19) United States
(12) Patent Application Publication
(10) Pub. No.: US 2004/0175418 A1
(43) Pub. Date: Sep. 9, 2004

(54) Teste masking pharmaceutical composition

(76) Inventors: Phillip John Ferguson, Kaiaua (NZ); Charles Robert Hillier, Auckland (NZ)

Correspondence Address: ECKERT SEAMANS CHERIN & MELLOTT, 600 GRANT STREET, 44TH FLOOR, PITTSBURGH, PA, 15219

(21) Appl. No.: 10/475,503

(22) PCT Filed: May 9, 2002
(86) PCT No.: PCT/NZ02/00091

(30) Foreign Application Priority Data
May 11, 2001 (NZ) 511657

(51) Int. Cl. A61K 31/7048; A61K 9/20

(52) U.S. Cl. 424/465; 514/28; 514/29

(57) ABSTRACT

The invention describes a composition suitable for oral administration comprising and antibiotic macrolide and a polycarbophil. The antibiotic macrolide is preferably clarithromycin. The polycarbophil is reported to have surprising taste-masking properties in combination with the antibiotic and acts by inhibiting the undesirable release of the antibiotic component in the mouth or stomach. Several methods of preparing granules of the antibiotic macrolide said polycarbophil are also described.

### FIELD

This invention relates to pharmaceutical compositions suitable for oral administration comprising polycarbophil and a beneficial agent. In particular it relates to compositions which allow for the controlled release of the beneficial agent for the purpose of masking its taste.

### BACKGROUND

Many prescription and non-prescription beneficial agents are known to have extremely unpleasant tastes. In particular the macrolide antibiotics, especially erythromycin and clarithromycin, have an extremely bitter taste making oral administration of these actives difficult. The administration of the macrolide antibiotics is often desirable in the treatment of children's ailments. As children cannot easily swallow tablets or capsules, it is preferable to provide them with medicaments in the form of suspensions or liquids. The extremely bitter taste of the above macrolide antibiotics makes this form of oral administration difficult to provide in that the children, and other patients, cannot tolerate the extremely unpleasant taste of the drug. There is therefore a need for palatable liquid dosage forms of beneficial agents and in particular the macrolide antibiotics.

### OBJECT

It is an object of the present invention to provide an oral composition which can deliver a pharmaco-kinetically acceptable dosage of a beneficial agent, or to at least provide the public with a useful choice.

### STATEMENT OF INVENTION

In a first aspect this invention provides a composition comprising a beneficial agent and polycarbophil.

In a second aspect this invention provides granules suitable for the preparation of liquid suspensions or dispersions for oral administration comprising a beneficial agent and polycarbophil.

Preferably the beneficial agent is a macrolide antibiotic, and in the most preferred compositions the beneficial agent is erythromycin, or a erythromycin derivative including clarithromycin.

In a third aspect this invention provides a process for the production of granules comprising a beneficial agent and polycarbophil, suitable for the preparation of liquid suspensions or dispersions for oral administration including the steps of:

blending the powders of polycarbophil and the beneficial agent in the required ratio;

adding a granulating fluid to the agitated blend to produce granules;

screening and drying the wet mass;

sizing the granules and collecting the preferred fraction.

Preferably the ratio of the beneficial agent and the polycarbophil is about 5:3

Preferably the granulating fluid is a solution of ethanol and water.

Preferably the granules are sized and regranulated with a binder solution.

Preferably the granules are coated in a polymeric coating.

The term macrolide antibiotic refers to a group of compounds having antibiotic activity and produced by streptomyces spp, characterised by having a macrocyclic ring, usually a 14-membered macrolactone ring and two clinked sugar molecules.

This particular ring system includes the erythromycins A, B, C and D. Especially useful macrolide antibiotics are erythromycin, clarithromycin, and roxithromycin.

The Erythromycins have the formulae:

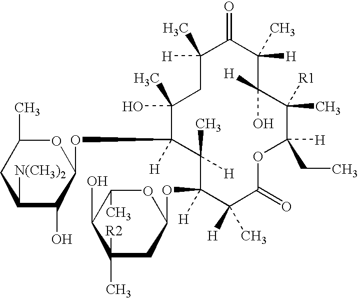

| Name | Mol. Formula | R1 | R2 |
|---|---|---|---|
| erythromycin A | C₃₇H₆₇NO₁₃ | OH | OCH₃ |
| erythromycin B | C₃₇H₆₇NO₁₂ | H | OCH₃ |
| erythromycin A | C₃₇H₆₅NO₁₃ | OH | OH |

and Clarithromycin has the formula

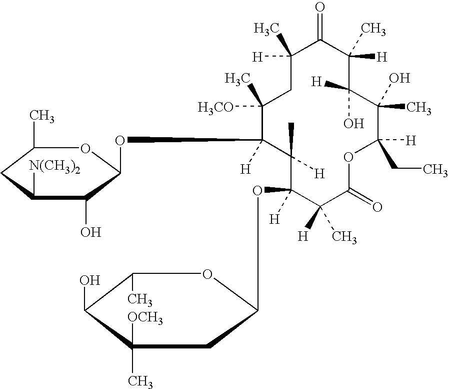

The compositions may also include the pharmaceutically acceptable salts and esters of the beneficial agent, or macrolide antibiotic.

Polycarbophil is a polymer of acrylic acid cross-linked with divinyl glycol. Polycarbophils are available through BF Goodrich as Noveon polycarbophils in both the calcium salt and acid forms. Polycarbophil is a synthetic agent that is not absorbed into the body. In the past it has been used to promote regular bowel activity and for the treatment of chronic constipation, diverticulosis and irratable bowel syndrome. In this capacity its main function is to absorb water in the intestine to create a bulkier and softer stool; it does not function as a laxative. For these purposes it is sold as an over the counter product under the trade names Fibercon, Equlactin and Mitrolan. Its use as a component in the preparation of taste-masking compositions for unpleasant or bitter-tasting beneficial agents such as the macrolide antibiotics has not previouly been known.

While the invention is not to be limited to any theory, it is thought that the following process may be involved in the ability of the polycarbophil polymer to facilitate the taste masking of the active. In its dehydrated state polycarbophil is believed to be a tightly coiled molecule. On hydration however, it uncoils slightly and consequently swells Partial neutralisation by the basic groups of the beneficial agent causes further uncoiling, swelling and entrapment of the beneficial agent, both physically and possibly chemically. When the beneficial agent is a macrolide antibiotic such as erythromycin or clarithromycin, some ionic bonding between the amine group of the antibiotic and the carboxyl groups of the polycarbophil may be present. Literature indicates that this chemical linkage exhibits optimum stability in the range pH 4 to 6 with dissolution of the antibiotic from the complex markedly increased at pH's outside this range. Because of this there is a possibility of an undesirable release of the active from the combination of antibiotic and polycarbophil in the acidic conditions of the stomach and neutral conditions of the mouth. In order to prevent premature release of the drug and any resultant unpalatability of the composition it is desirable to provide the granules with an acid resistant coating. This protective coat allows rapid release of the drug in the higher pH environment of the duodenum and through the intestinal tract. Thus release of the antibiotic from the coated combination of antibiotic and polycarbophil is inhibited until after the composition has passed through the mouth and stomach, therefore eliminating any of the tasting of the active by the patient.

By inhibiting the release of the active from the composition in the mouth and stomach the compositions of the invention provide palatable oral dosage forms of the antibiotics while maintaining acceptable pharmacokinetic properties. The polycarbophil is not absorbed into the body, and it is known from previous applications in the treatment of constipation to be safe for long term use.

Preferably the compositions of the invention are provided as granules to be used in the preparation of aqueous suspensions or dispersions. However, it is envisaged as being within the scope of the invention that the granules maybe employed in the preparation of other known dosage forms such as tablets, capsules and chewable preparations.

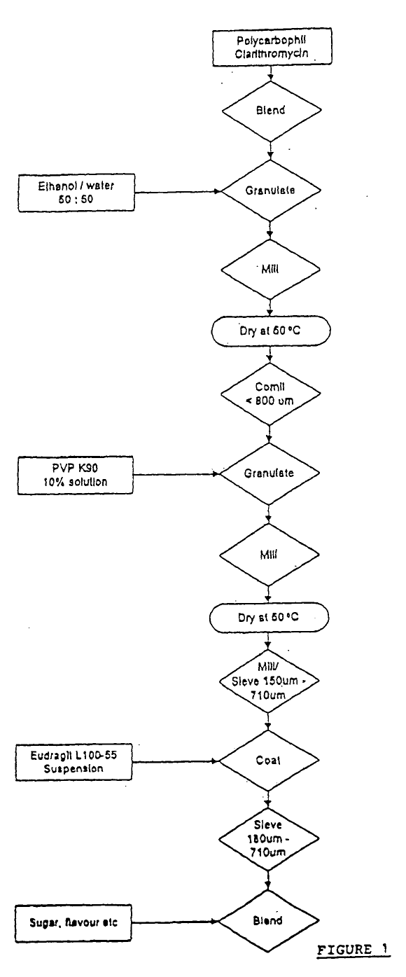

A preferred process for the production of the granules will be described by way of example only with reference to the flow diagram of FIG. 1.

A selected ratio of the beneficial agent and polycabophil powders are thoroughly blended. The preferred ratio of the powders is about 5:3 when the active ingredient is a macrolide antibiotic although any ratio which produces a therapeutically effective product is envisaged as being within the scope of this invention.

Any standard pharmaceutical blender may be used eg a planetary mixer has been found to be particularly suitable. Once the powders are blended a granulating solution of alcohol and water is added to the agitated blend over a period of about 1 hour to allow the granules to form. The head space temperature is maintained at below 60° C. The preferred granulating solution comprises ethanol in water in equal amounts by weight. It has been found that if only water is used as the granulating liquid the wet mass tends to granulate more rapidly and lump making granulation less satisfactory. The introduction of ethanol into the granulating solution slows down the process of gelation/granulation and gives improved granules. The resultant wet mass is screened and dried to LOD<4%. The preferred drying temperature is 50° C. The dried mass is milled to a particle size of less than 800 μm. While the granules may be used at this stage it has been found that it is preferable to coat the resulting granules with a polymeric coating and preferably prior to this step, to size and re-granulate with a binder solution. The sized granules are preferably regranulated with a 10% aqueous Povidone K90 binder solution. The resultant wet mass is screened and dried at 50° C. to LOD<4%. The dried material is then milled and sieved to recover the fraction between 180 μm and 710 μm. The collection fraction is coated with a suitable aqueous enteric coating to enhance the taste masking function and the preferred material in this regard is Budagrit L100-55 suspension. The granules are coated by fluidising in a fluid bed apparatus and spraying them with the coating suspension, although any of the well known methods for coating granules may be employed. The coated granules are then re-sieved to recover the fraction between 180 μm and 710 μm. The finished granules may be mixed with sweeteners, flavouring agents, preservatives or any other ingredients which when dispersed in water provide a therapeutic composition suitable for oral administration. Preferably the resulting dispersion will be suitable for paediatric administration.

Some preferred compositions are detailed in the following examples, in which dissolution data is provided for Examples 4, 5 and 6. However it will be appreciated that the invention is not to be limited to these examples.

### EXAMPLE 1

Clarithromycin (83.3 g) and polycarbophil (50 g) were thoroughly blended together for 10 minutes in the mixing bowl of a planetary mixer. Ethanol (212 g) was slowly added to the powders whilst mixing over a period of 15 minutes. Mixing was continued for 10 minutes. The wet mass was dried at 50° C. The dried granule was passed through a Comil fitted with a 800 μm screen. A second granulation was carried out using the previously processed granule and a 10% aqueous solution of polyvinyl pryrrolidone (PVP) K90 (45 g). The wet mass was dried at 50° C. for 18 hours and then milled, sieved and the fraction 180-500 μm collected. The finished granule was robust and although the taste was slightly bitter a larger batch, when coated, may possess the desired organoleptic qualities.

### EXAMPLE 2

Clarithromycin (75 g) and polycarbophil (45 g) were thoroughly blended together in the mixing bowl. Whilst stirring the blend a solution of PVP K90 (6.6 g) in ethanol (66.6 g) was added to form a wet mass. The wet mass was dried at 50° C. for 15 hours and then milled and sieved. The resultant granule was robust but as before the taste was unsatisfactory.

### EXAMPLE 3

Clarithromycin (50 g) and polycarbophil (30 g) were thoroughly blended together in the mixing bowl. Granulating fluid comprising Ethanol and purified water in the ration 50:50 was added to the mixing powders over a period of 1 hour to form a wet mass. The wet mass was milled to provide a suitable texture for drying. After drying at 50° C. the granule was milled through a 800 μm screen and regranulated with a 10% w/w aqueous solution of PVP K90 (50 g). Again the wet mass was dried at 50° C. until the LOD<3%. The dried granule was milled and sieved with the fraction 180-500 μm retained. Although the finished granule possessed a residual bitter aftertaste, the ethanol/purified water granulating fluid allowed for a smoother initial granulating process.

### EXAMPLE 4

Clarithromycin (375 g) and Polycarbophil (225 g) were thoroughly blended in the mixing bowl. The blended powders were granulated using ethanol/purified water (50:50) (800 g) over a period of 1 hour. As per previous examples the wet mass was dried and sized prior to a second granulation with 10% w/w aqueous PVP K90 solution (316 g). The fraction (180-710 μm) collected after milling and sieving was coated with Eudragit L 100-55 in a fluid bed apparatus using the bottom spray technique in the Wurster mode. When tested in dissolution mediuri at pH 6.8 the prepared granule exhibited a satisfactory dissolution profile. The taste characteristic of the granule blended with other excipients and reconstituted with water was satisfactory, the bitterness of clarithromycin being masked for a 14 day storage period.

Assay—Bottom 249 mg/g

Dissolution—Simulated Gastric Fluid

| | Time(min) | | | | | | |
|---|---|---|---|---|---|---|---|
| | 0 | 30 | 60 | 90 | 120 | 180 | 240 |
| % Dissolved | 0.0 | 0.0 | 0.0 | 0.0 | 0.0 | 0.0 | 0.0 |

Dissolution—Phosphate Buffer pH 6.8

| | Time(min) | | | | |
|---|---|---|---|---|---|
| | 0 | 15 | 30 | 45 | 60 |
| % Dissolved | 0.0 | 46.4 | 82.7 | 96.0 | 101.1 |

### EXAMPLE 5

Clarithromycin (750 g) and polycarbophil (450 g) were blended and divided into 4 equal portions. Each portion was granulated with a blend of ethanol/purified water (50:50) (350 g). The granulating fluid was added at a rate of approximately 10 ml/minute with continuous mixing. The combined wet masses were then processed as per the attached chart The granule was split into two portions prior to fluid bed coating. One portion was coated by the bottom spray technique whilst the other portion was coated by the top spray technique. Both techniques yielded a useable granule possessing a good taste masking characteristic. In both cases a certain degree of secondary granulation was noted during the coating process which would require optimisation.

| | |
|---|---|
| Assay - Bottom | 247 mg/g |
| Top | 289 mg/g |

Dissolution—Phosphate Buffer pH 6.8

| | | Time(min) | | | | |
|---|---|---|---|---|---|---|
| | Sample | 0 | 15 | 30 | 45 | 60 |
| % Dissolved | Bottom | 0.0 | 5.3 | 18.3 | 30.7 | 38.6 |
| | Top | 0.0 | 4.5 | 12.8 | 21.9 | 29.8 |

### EXAMPLE 6

A thoroughly mixed blend of clarithromycin (750 g) and polycarbophil (450 g) was granulated as per the attached flow chart using ethanol/water (1.3 kg) added over a period of 1 hour and subsequently 10% PVP K90 (635 g). During processing the product temperature was monitored to ensure that 60° C. was not exceeded. The finished granule was tested for moisture content which averaged 3.8% (LOD). As part of the coating process using the top spray technique samples were removed periodically to evaluate the ability of differing levels of coat to mask the bitter taste. It was found that taste masking was effective after 386 g of Eudragit L 100-55 polymer had been applied.

| Assay - 1 | 341 mg/g |
|---|---|
| 2 | 290 mg/g |
| 3 | 250 mg/g |
| 4 | 238 mg/g |

Dissolution—Phosphate Buffer pH 6.8

| | | Time(min) | | | | |
|---|---|---|---|---|---|---|
| | Sample | 0 | 15 | 30 | 45 | 60 |
| % | 1 | 0.0 | 47.8 | 78.1 | 87.5 | 89.9 |
| Dissolved | 2 | 0.0 | 46.4 | 84.1 | 93.3 | 94.3 |
| | 3 | 0.0 | 43.6 | 87.0 | 100.2 | 103.7 |
| | 4 | 0.0 | 36.6 | 83.6 | 97.7 | 101.5 |

Throughout the description and claims of this specification the word “comprise” and variations of that word such as “comprises” and “comprising” are not intended to exclude other additives, components, integers or steps.

1 an oral pharmaceutical composition comprising a macrolide and a polycarbophil.

2. The oral pharmaceutical composition according to claim 1 wherein the macrolidc is selected from the group comprising eromycin A, crythromycin B, erythromycin C, erythromycin D, clarithromycin, dirithromycin, josamycin, midecamnycin, kitasamycin, tylosin, roxithromycin, rokitamycin, oleandomycin, miocamycin, flunthromycin, rosarmicin spiramycin and azithrornycin.

3. The oral pharmaceutical composition of claim 1 wherein the macrolide is clarithromycin.

4. The oral pharmaceutical composition according to claim 1 or claim 2 wherein the weight ratio of macrolide to polycarbophil is between about 1:10 and about 5:1.

5. The oral pharmaceutical composition according to claim 1 or claim 2 wherein the weight ratio of macrolide to polycarbophil is between about 1:2 and about 5:2.

6. The oral pharmaceutical composition of claim 1 or claim 2 wherein the weight ratio of macrolide to polycarbophil is about 5:3

7. The oral pharmaceutical composition of any one of claims 1-6 comprising an ionic complex of macrolidc and polycarbophil.

8. The oral pharmaceutical composition of any one of claims 1-7 in a granular form suitable for the preparation of liquid suspensions or dispersions or for formulating into chewable tablets.

9. A process for preparing granules of a macrolide and a polycarbophil comprising the steps of:
(i) mixing a macrolidc and a polycarbophil in a weight ratio of macrolide to polycarbophil is between about 1:10 and about 5:1,
(ii) wetting the mixture in a granulating solution,
(iii) blending the wetted mixture for a time sufficient to form granules in a blender wherein the head space temperature is maintained at below 60° C., and
(iv) drying and screening the resultant dried mass to form the desired macrolide-polycarbophil granules.

10 The process according to claim 9 further comprising a second granulation procedure, the procedure comprising the steps of:
(v) sizing the dried granules prepared at step (iv) with a suitable binder solution such as a 10% aqueous polyvinyl pyrrolidone (PVP) K90, and
(vi) drying and, milling or serving the dried mass to recover granules with a particle size of between 180μ and 710μ and optionally
(vii) coating the granules with a suitable enteric coating.

11. The process of claim 9 or claim 10 wherein the macrolide is selected from the group comprising erythromycin A, erythromycin B, erythromycin C, erythromycin D, clarithromycin, dirithromycin, josamycin, midecamycin, kitasamycin, tylosin, roxithromycin, rokitamycin, oleandomycin, miocamycin, flurithromycin, rosanrnicin and azithromycin.

12. The process of claim 11 wherein the macrolide is clarithromycin.
